(19) United States
(12) Patent Application Publication
(10) Pub. No.: US 2021/0231682 A1
(43) Pub. Date: Jul. 29, 2021

(54) COMPOSITIONS AND METHODS FOR TREATING VASCULAR DISEASE IN SELECTED PATIENTS

(71) Applicant: The University of Vermont and State Agriculture College, Burlington, VT (US)

(72) Inventor: David Schneider, Burlington, VT (US)

(73) Assignee: The University of Vermont and State Agriculture College, Burlington, VT (US)

(21) Appl. No.: 17/048,926

(22) PCT Filed: Apr. 16, 2019
(86) PCT No.: PCT/US2019/027684

§ 371 Date: Oct. 19, 2020

Related U.S. Application Data
(60) Provisional application No. 62/660,627, filed on Apr. 20, 2018.

(51) Int. Cl. G01N 33/68; A61K 45/06; G01N 33/50

(52) U.S. Cl. CPC G01N 33/6893; A61K 45/06; G01N 2496/00; G01N 33/6857; G01N 2800/32; G01N 33/5094

(57) ABSTRACT

As described below, the present invention features methods for treating selected subjects at increased risk of a cardiovascular event, wherein the subjects are selected as having elevated platelet Fcγgamma RIIa.

### CROSS-REFERENCE TO RELATED APPLICATION

This application claims the benefit of the following U.S. Provisional Application No. 62/660,627, filed Apr. 20, 2018, the entire contents of which are incorporated herein by reference.

### BACKGROUND OF THE INVENTION

Increased platelet reactivity that has been identified with multiple assays has been associated with a greater risk of subsequent cardiovascular events. Despite a consistent association between increased platelet reactivity and greater risk, individualized antiplatelet therapy guided by platelet function testing has not been shown to reduce the subsequent risk of cardiovascular events. Patients undergoing percutaneous coronary intervention (PCI) with high on-treatment platelet reactivity who were randomized to high dose clopidogrel had a similar incidence of cardiovascular events compared with those on standard dose clopidogrel. A subsequent study in which patients with high on-treatment platelet reactivity were randomized to more powerful antiplatelet therapy and in whom suppression of platelet function was confirmed did not demonstrate that intensification of antiplatelet therapy in patients with high on treatment platelet reactivity reduced their subsequent risk of cardiovascular events.

Assays used to identify high platelet reactivity require activation of platelets in vitro. Preparative procedures such as the method of phlebotomy, the anticoagulant, and the concentration of agonist influence the assessment of platelet reactivity. Moreover platelet function tests exhibit substantial intra-individual variability, even during the course of one day. Intra-individual variability in platelet function and its potential influence on the classification of patients as exhibiting high platelet reactivity has been identified as a potential contributor to the lack of efficacy associated with individualized antiplatelet therapy based on the results of platelet function testing. Improved methods for identifying subjects at risk for cardiovascular events and selecting appropriate therapies are urgently required.

### SUMMARY OF THE INVENTION

As described below, the present invention features methods for treating subjects identified as having elevated platelet FcγRIIa that places them at increased risk for a cardiovascular event, as well as methods for identifying subjects in need of such treatment.

In one aspect, the invention generally provides a method of treating a selected subject (e.g., human) at increased risk of a cardiovascular event (e.g., myocardial infarction, stroke, coronary revascularization, cerebral vascular accident, death, or another cardiovascular endpoint), the method involving administering to the subject an anti-inflammatory agent, wherein the subject is selected by detecting an increased level of FcγRIIa on platelets from the subject relative to a reference.

In another aspect, the invention provides a method of treating a selected subject having systemic inflammation, the method involving administering to the subject an anti-inflammatory agent, wherein the subject is selected by detecting an increased level of FcγRIIa on platelets from the subject relative to a reference.

In another aspect, the invention provides a method of identifying a subject at increased risk of a cardiovascular event involving:

determining a level of FcγRIIa on platelets from the subject; and

comparing the level of FcγRIIa on the platelets with a reference value, wherein an increased level compared to the reference value indicates that the subject is at increased risk of a cardiovascular event.

In another aspect, the invention provides a method of identifying a subject at increased risk of a cardiovascular event involving:

determining a level of FcγRIIa on platelets from the subject; and

comparing the level of FcγRIIa on the platelets with a reference value wherein an increased level compared to the reference value indicates that the subject is at increased risk of a cardiovascular event.

In another aspect, the invention provides a method of identifying a subject as having systemic inflammation involving:

determining a level of FcγRIIa on platelets from the subject; and

comparing the level of FcγRIIa on the platelets with a reference value wherein an increased level compared to the reference value indicates that the subject has systemic inflammation.

In various embodiments of any of the above aspects, the method further involves characterizing a biological sample of the subject for the level of IL-1beta, TNF-alpha, IL-6, C reactive protein (CRP), and/or serum amyloid A. In various embodiments of any of the above aspects, the level of FcγRIIa is determined using an FcγRIIa specific reagent (e.g., an antibody or antigen-binding fragment thereof). In other embodiments, the level of platelet FcγRIIa is determined using an assay that is flow cytometry, immunoassay, ELISA, western blotting, or radioimmunoassay. In other embodiments, the level of FcγRIIa is determined using fluorometric or colorimetric assay. In other embodiments, the level of FcγRIIa is determined using flow cytometry. In other embodiments, the reference value represents a level of FcγRIIa on platelets from disease-free subjects. In other embodiments, the increased level is increased by at least about 1.5, 2, 3, 4, or 5-fold. In other embodiments, the reference level of platelet FcγRIIa expression is less than about 11,000/platelet. In other embodiments, the reference level of platelet FcγRIIa expression is less than about 7,500, 8,000, 9,000, 10,000, or 10,500 copies of FcγRIIa per platelet. In other embodiments, the increased level of platelet FcγRIIa expression is greater than about 11,000 copies of FcγRIIa per platelet. In other embodiments, the increased level is between about 11,000-20,000 copies of FcγRIIa per platelet. In other embodiments, the increased level is between about 11,000-15,000 copies of FcγRIIa per platelet. In other embodiments of the above aspects, the method further involves administering an anti-thrombotic therapy (e.g., prasugrel, ticagrelor, clopidogrel, and vopaxar) to the selected subject. In other embodiments of the above aspects, the method further involves administering an anti-coagulant therapy (e.g., rivaroxaban or warfarin) to the selected subject. In various embodiments of the above aspects, the cardiovascular event is myocardial infarction, stroke, coronary revascularization, cerebral vascular accident, death, or another cardiovascular endpoint. In various embodiments of the above aspects, the agent is methotrexate or canakinumab. In various embodiments of the above aspects, the cardiovascular event is myocardial infarction, stroke, coronary revascularization, cerebral vascular accident, death, or another cardiovascular endpoint.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “cardiovascular event” is meant any acute pathological condition associated wan the cardiovasculature. Cardiovascular events include, but are not limited to, myocardial infarction, coronary revascularization, cerebral vascular accident, and death.

By “inflammation” is meant a condition associated with an increase in pro-inflammatory cytokines and/or other markers of inflammation, including IL-1beta, TNF-alpha, IL-6, C reactive protein (CRP), and/or serum amyloid A.

By “platelet reactivity” is meant the sensitivity of platelets to activation and clotting.

By “FcγRIIa” is meant the low affinity immunoglobulin gamma Fc region receptor II-a. An illustrative amino acid sequence of the FcγRIIa is provided at GenBank Accession No. NP_001129691.1″

| | |
|---|---|
| 1 | mtmetqmsqn vcprnlwllq pltvllllas adsqaaappk avlkleppwi nvlqedsvtl |
| 61 | tcqgarspes dsiqwfhngn lipthtqpsy rfkannndsg eytcqtgqts lsdpvhltvl |
| 121 | sewlvlqtph lefqegetim lrchswkdkp lvkvtffqng ksqkfshldp tfsipqanhs |
| 181 | hsgdyhctgn igytlfsskp vtitvqvpsm gssspmgiiv avviatavaa ivaavvaliy |
| 241 | crkkrisans tdpvkaaqfe ppgrqmiair krqleetnnd yetadggymt lnpraptddd |
| 301 | kniyltlppn dhvnsnn. |

An illustrative nucleic acid sequence encoding FcγRIIa is provided at GenBank Accession No. NM_001136219.1:

ATGACTATGGAGACCCAAATGTCTCAGAATGTATGTCCCAGAAACCTGTG
GCTGCTTCAACCATTGACAGTTTTGCTGCTGCTGGCTTCTGCAGACAGTC
AAGCTGCAGCTCCCCCAAAGGCTGTGCTGAAACTTGAGCCCCCGTGGATC
AACGTGCTCCAGGAGGACTCTGTGACTCTGACATGCCAGGGGGCTCGCAG
CCCTGAGAGCGACTCCATTCAGTGGTTCCACAATGGGAATCTCATTCCCA
CCCACACGCAGCCCAGCTACAGGTTCAAGGCCAACAACAATGACAGCGGG
GAGTACACGTGCCAGACTGGCCAGACCAGCCTCAGCGACCCTGTGCATCT
GACTGTGCTTTCCGAATGGCTGGTGCTCCAGACCCCTCACCTGGAGTTCC
AGGAGGGAGAAACCATCATGCTGAGGTGCCACAGCTGGAAGGACAAGCCT
CTGGTCAAGGTCACATTCTTCCAGAATGGAAAATCCCAGAAATTCTCCCA
TTTGGATCCCACCTTCTCCATCCCACAAGCAAACCACAGTCACAGTGGTG
ATTACCACTGCACAGGAAACATAGGCTACACGCTGTTCTCATCCAAGCCT
GTGACCATCACTGTCCAAGTGCCCAGCATGGGCAGCTCTTCACCAATGGG
GATCATTGTGGCTGTGGTCATTGCGACTGCTGTAGCAGCCATTGTTGCTG
CTGTAGTGGCCTTGATCTACTGCAGGAAAAAGCGGATTTCAGCCAATTCC
ACTGATCCTGTGAAGGCTGCCCAATTTGAGCCACCTGGACGTCAAATGAT
TGCCATCAGAAAGAGACAACTTGAAGAAACCAACAATGACTATGAAACAG
CTGACGGCGGCTACATGACTCTGAACCCCAGGGCACCTACTGACGATGAT
AAAAACATCTACCTGACTCTTCCTCCCAACGACCATGTCAACAGTAATAA
CTAA.

By “FcγRIIa specific agent” is meant any small molecule compound, antibody, nucleic acid molecule, or polypeptide, or fragments thereof that specifically bind to FcγRIIa.

By “Protease-activated receptor (PAR)” is meant a G protein-coupled receptor that is activated by cleavage of a portion of its extracellular domain. PARs are highly expressed in platelets, including the thrombin receptors PAR1, PAR3 and PAR4. PARs are activated by the action of serine proteases such as thrombin (e.g., activating PARs 1, 3 and 4). Cleavage of the N-terminus of the receptor, generates a tethered ligand (SFLLRN) that acts as an agonist, causing a physiological response. The cellular effects of thrombin are mediated by protease-activated receptors (PARs). Thrombin signaling in platelets contributes to hemostasis and thrombosis. Thrombin receptor antagonists include Vorapaxar (SCH 530348) which is a PAR1 antagonist.

By “Adenosine diphosphate (ADP) receptor” is meant a purinergic G protein-coupled receptors, stimulated by the nucleotide Adenosine diphosphate (ADP). ADP receptors include P2Y12 which regulates thrombosis. Adenosine diphosphate (ADP) receptor antagonists are agents that inhibit adenosine diphosphate receptors. P2Y12 is the target of the anti-platelet drugs including prasugrel, clopidogrel, and other thienopyridines.

By “clopidogrel” is meant (+)-(S)-methyl 2-(2-chlorophenyl)-2-(6,7-dihydrothieno[3,2-c]pyridin-5(4H)-yl)acetate which is a potent platelet aggregation inhibitor.

By “prasugrel” is meant (RS)-5-[2-cyclopropyl-1-(2-fluorophenyl)-2-oxoethyl]-4,5,6,7-tetrahydrothieno[3,2-c]pyridine-2-yl acetate which is a potent platelet aggregation inhibitor.

By “ticagrelor” is meant (1S,2S,3R,5S)-3-[7-[(1R,2S)-2-(3,4-Difluorophenyl)cyclopropylamino]-5-(propylthio)-3H-[1,2,3]triazolo[4,5-d]pyrimidin-3-yl]-5-(2-hydroxyethoxy)cyclopentane-1,2-diol which is a potent platelet aggregation inhibitor.

By “vorapaxar” is meant Ethyl N-[(3R,3aS,4S,4aR,7R,8aR,9aR)-4-[(E)-2-[5-(3-fluorophenyl)-2-pyridyl]vinyl]-3-methyl-1-oxo-3a,4,4a,5,6,7,8,8a,9,9a-decahydro-3H-benzo[f]isobenzofuran-7-yl]carbamate which is a potent platelet aggregation inhibitor.

By “anti-thrombotic therapy” is meant any treatment used to inhibit platelet aggregation in a subject.

By “agent” is meant any small molecule chemical compound, antibody, nucleic acid molecule, or polypeptide, or fragments thereof. In particular embodiments, an agent is an agent that inhibits inflammation, particularly inflammation associated with a cardiovascular event, or an agent that inhibits platelet aggregation. In one embodiment, a the agent is methotrexate or canakinumab. Such anti-inflammatory agents can be used alone or in combination with an anti-thrombotic therapy.

By “ameliorate” is meant decrease, suppress, attenuate, diminish, arrest, or stabilize the development or progression of a disease. Disease amenable to treatment using the methods of the invention include inflammatory cardiovascular diseases (e.g., myocardial infarction, stroke, coronary revascularization, cerebral vascular accident, death, or other cardiovascular endpoints).

By “alteration” is meant a change (increase or decrease) in the expression levels or activity of an analyte (e.g., marker gene or polypeptide) as detected by standard art known methods such as those described herein. As used herein, an alteration includes a 10% change in expression levels, preferably a 25% change, more preferably a 40% change, and most preferably a 50% or greater change in expression levels. “

By “analyte” is meant any agent under investigation using an analytical method.

By “analyte-binding conjugate” is meant a detectable molecule that binds a compound under investigation.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “a control conjugate” is meant a detectable molecule that does not substantially bind a compound under investigation.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include systemic inflammation or cardiovascular inflammation, as well as thrombotic disease associated wan an undesirable increase in platelet reactivity and/or the formation of a thrombus, such as a thrombus that results in an ischemic event.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount reduces systemic inflammation and/or reduces the level of a marker of disease (e.g., marker of inflammation, such as IL-1, IL-6, CRP or marker of cardiovascular risk, such as FcγRIIa).

The invention provides a number of targets that are useful for the development of highly specific drugs to treat a cardiovascular disease or disorder characterized by the methods delineated herein (e.g., characterized by an undesirable increase in platelet reactivity). In addition, the methods of the invention provide a facile means to identify therapies that are safe for use in subjects. In addition, the methods of the invention provide a route for analyzing virtually any number of compounds for effects on a cardiovascular disease described herein with high-volume throughput, high sensitivity, and low complexity, as well as for treating such diseases.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. In one embodiment, the marker is FcγRIIa level, activity, phosphorylation, or expression, and an increase in said marker is associated with an increased propensity to have a cardiovascular event (e.g., myocardial infarction, stroke, coronary revascularization, cerebral vascular accident, death, or other cardiovascular endpoints), systemic inflammation, and/or increased platelet reactivity. In other embodiments, the marker is a marker of inflammation (e.g., IL-1beta, TNF-alpha, IL-6, C reactive protein (CRP), and/or serum amyloid A).

By “portion” is meant some fraction of a whole.

As used herein, “obtaining” as in “obtaining an agent” includes synthesizing, purchasing, or otherwise acquiring the agent.

By “reduces” is meant a negative alteration of at least 10%, 25%, 50%, 75%, or 100% in a parameter.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A is a flow chart that delineates the results described herein.

FIG. 1B shows Kaplan-Meier curves of the probability of freedom from cardiovascular events (myocardial infarction, coronary revascularization, cerebral vascular accident, and death). Platelet expression of FcγRIIa was quantified with the use of flow cytometry. The average duration of follow-up was 20 months (range 6-29 months).

FIG. 2 is a bar graph shown changes in platelet expression of FcγRIIa between baseline and 6 months. The average platelet expression of FcγRIIa was 11,479/platelet at baseline (n=197) and 10,643/platelet at 6 months (n=114). Individual changes in the platelet expression of FcγRIIa were quantified as a percentage of the average for all patients. The distribution of relative change is shown.

FIG. 3 is a scatter plot showing the relationship between platelet expression of FcγRIIa at baseline and at 6 months. Platelet expression of FcγRIIa was quantified with the use of flow cytometry. Baseline expression of FcγRIIa predicted expression at 6 months (r=0.67, p<0.001).

### DETAILED DESCRIPTION OF THE INVENTION

The invention features compositions and methods that are useful for treating a selected subject at increased risk of a cardiovascular event (e.g., myocardial infarction, stroke, coronary revascularization, cerebral vascular accident, death, or other cardiovascular endpoints), and methods for selecting such patients. In particular embodiments, a subject having increased FcγRIIa is treated with powerful antiplatelet agents, anticoagulants, and anti-inflammatory agents.

The invention is based, at least in part, on the findings that increased platelet expression of FcγRIIa identifies patients at greater risk of subsequent a cardiovascular event and that platelet expression of FcγRIIa provides a tangible link between systemic inflammation and increased platelet reactivity. As reported in greater detail below, 197 patients were enrolled after a myocardial infarction (MI) before discharge. Platelet expression of FcγRIIa was quantified with the use of flow cytometry at enrollment and at 6±1 months. CV (MI, coronary revascularization, cerebral vascular accident [CVA], and death) as well as bleeding were assessed after 6 months, 1 year, and at the end of study.

Patients with platelet expression of FcγRIIa >11,000/platelet had an increased incidence of CV (17.3% vs 6.5%, odds ratio [OR] 3.2, 95% confidence interval [CI] 1.23-8.51, p=0.017). The increase in CV was driven by a greater incidence of MI, CVA, and death (3.2% vs 12.5% OR 4.3, 95% CI 1.18-15.6, p=0.027). Bleeding was similar in the 2 groups. All patients were treated with aspirin and treatment with clopidogrel (˜64%) and ticagrelor (˜36%) was balanced in patients with high and low platelet expression of FcγRIIa. Cox multivariate analysis demonstrate a hazard ratio 3.0 (p=0.02) for platelet expression of FcγRIIa >11,000 when age, diabetes, and prior revascularization were included as covariates.

Accordingly, the invention provides methods for treating selected patients having elevated FcγRIIa that places them at increased risk of having a cardiovascular event. The subject is selected for treatment by determining platelet reactivity in a biological sample of a subject. In particular, an increase in FcγRIIa is useful in identifying a subject that could benefit from treatment with anti-inflammatory, such as canakinumab or methotrexate. Such anti-inflammatory therapy may be administered alone or in combination with an anti-platelet agent, such as Vorapaxar, Clopidogrel, Prasugrel or Ticagrelor, and/or an anticoagulant, such as rivaroxaban or warfarin.

### Therapeutic Methods

The present invention features therapies for subjects selected as having elevated platelet FcγRIIa that places them at increased risk of a cardiovascular event (e.g., myocardial infarction, coronary revascularization, cerebral vascular accident, and death). In particular, the method provides for the administration of an anti-inflammatory agent (e.g., canakinumab or methotrexate) to subjects selected as having an increased level of FcγRIIa. Because platelet expression of FcG is increased by inflammation, elevated platelet FcγRIIa that identifies patients at increased risk for cardiovascular events will also identify patients who would potentially benefit from anti-inflammatory therapy.

### FcγRIIa Measurement

Methods for measuring FcγRIIa are known in the art and described herein. In one embodiment, levels of platelet FcγRIIa are measured in a subject sample and used to characterize cardiovascular risk in the subject. Any suitable method can be used to detect platelet FcγRIIa in a subject biological sample. Biological samples include bodily fluids (e.g., blood, blood serum, plasma, and saliva), which are analyzed using virtually any method that detects and/or quantifies platelet FcγRIIa. Such methods include immunoassays in various formats (e.g., flow cytometry, ELISA), which are popular methods for detection of analytes captured on a solid phase. Such methods typically involve use of an FcγRIIa specific antibody.

Levels of platelet FcγRIIa are compared by procedures well known in the art, such as flow cytometry, immunoassay, ELISA, western blotting, radioimmunoassay, immunocytochemistry, binding to magnetic and/or antibody-coated beads, in situ hybridization, fluorescence in situ hybridization (FISH), flow chamber adhesion assay, microarray analysis, or colorimetric assays. Methods may further include, one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS)ₙ, quadrupole mass spectrometry, fourier transform mass spectrometry (FTMS), and ion trap mass spectrometry, where n is an integer greater than zero.

Detection methods may include use of a biochip array. Biochip arrays useful in the invention include protein and polynucleotide arrays. One or more markers are captured on the biochip array and subjected to analysis to detect the level of the markers in a sample.

Platelet FcγRIIa may be captured with capture reagents fixed to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltammetry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapour phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Frazingham, Mass., USA) and are described in the literature, e.g. M. Kussmann and P. Roepstorff, cited above.

In other embodiments, levels of FcγRIIa are detected in combination with one or more additional markers (e.g., markers of inflammation, such as IL-1beta, TNF-alpha, IL-6, C reactive protein (CRP), and/or serum amyloid A). While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, methods of the present invention provide for the measurement of more than one marker or clinical parameter.

The use of multiple markers increases the predictive value of the test and provides greater utility in diagnosis, toxicology, patient stratification and patient monitoring. The process called “Pattern recognition” detects the patterns formed by multiple markers. The inclusion of additional markers may improve the sensitivity and specificity in determining a patient's risk of a cardiovascular event (e.g., myocardial infarction, stroke, coronary revascularization, cerebral vascular accident, death, or other cardiovascular endpoints). Subtle variations in data from clinical samples indicate that certain patterns of protein level or expression (e.g., FcγRIIa level) predict subject risk of having a cardiovascular event. Patients having elevated levels of FcγRIIa, e.g., greater than about 11,000 copies per platelet, will benefit from treatment with methotrexate or canakinumab, alone or in combination with a more powerful anti-platelet agent, such as Prasugrel or Ticagrelor, and/or an anticoagulant (e.g., rivaroxaban, warfarin).

Antibodies that specifically bind FcγRIIa, or any other method known in the art may be used to monitor expression of platelet FcγRIIa. Detection of an alteration relative to a normal, reference sample can be used as a diagnostic indicator of systemic inflammation. In particular embodiments, a 2, 3, 4, 5, or 6-fold change in the level of platelet FcγRIIa is indicative of systemic inflammation.

In one embodiment, the level of platelet FcγRIIa is measured on at least two different occasions and an alteration in the levels as compared to normal reference levels over time is used as an indicator of platelet reactivity or the propensity to have a cardiovascular event. In general, levels of platelet FcγRIIa are present at low levels (less than about 8,000 copies/platelet or less than about 11,000 copies per platelet (e.g., 5,000, 6,000, 7,000, 8,000, 9,000, 10,000 copies per platelet) in a healthy subject. In one embodiment, elevated levels of platelet FcγRIIa equal to or greater than about 11,000 copies per platelet (e.g., 11,000, 12,000, 13,000, 14,000, 15,000, 16,000, 17,000, 18,000, 19,000, or 20,000 copies per platelet) is indicative of systemic inflammation and/or risk of a cardiovascular event. In another embodiment the increased level is about 11,100, 11,200, 11,300, 11,400, 11,500, 11,600, 11,700, or from about 11,000 to 15,000 copies per platelet. In one embodiment, FcγRIIa copy/platelet is measured using FACS analysis.

The correlation may take into account the amount of platelet FcγRIIa in the sample compared to a control amount of platelet FcγRIIa (e.g., in normal subjects or in subjects where platelet reactivity is undetected). A control can be, e.g., the average or median amount of platelet FcγRIIa present in comparable samples of normal subjects. The control amount is measured under the same or substantially similar experimental conditions as in measuring the test amount. As a result, the control can be employed as a reference standard, where the normal phenotype is known, and each result can be compared to that standard, rather than re-running a control.

Accordingly, a marker profile may be obtained from a subject sample and compared to a reference value obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of cancer status.

In certain embodiments, the methods further comprise selecting anti-inflammatory therapy. For example, where a 2-5 fold, 5-10 fold, or 10-25 fold increase in platelet FcγRIIa levels or levels greater than about 11,000 copies/platelet relative to a reference identifies a patient that could benefit from treatment with methotrexate or canakinumab alone or in combination with treatment with an anti-platelet agent, such as Prasugrel or Ticagrelor; and/or treatment with an anticoagulant, such as rivaroxaban or warfarin. The invention also provides for methods where platelet FcγRIIa is measured again after therapy.

### Antibodies

As reported herein, antibodies that specifically bind FcγRIIa are useful in diagnostic, as well as therapeutic methods. For example, antibodies that act as platelet FcγRIIa antagonists (e.g., IV.3 Fab) are particularly useful in the methods of the invention. In particular embodiments, the invention provides methods of using anti-platelet FcγRIIa antibodies for the inhibition of platelet reactivity. IV.3 is a monoclonal anti-FcγRIIa antibody that inhibits the phosphorylation of platelet FcγRIIa during platelet activation.

Other antibodies useful in the invention are those that attenuate platelet FcγRIIa signaling. Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of an intact antibody, clear more rapidly from the circulation, and may have less non-specific tissue binding than an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of 60-100 kDa in size provide faster blood clearance and rapid tissue uptake. See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that binds platelet FcγRIIa is monoclonal. Alternatively, the anti-platelet FcγRIIa antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab”′ fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing soluble polypeptides, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding human FcγRIIa or immunogenic fragments thereof, can be provided to the host in a delivery vehicle that is taken up by immune cells of the host. The cells will in turn express the human FcγRIIa thereby generating an immunogenic response in the host. Alternatively, nucleic acid sequences encoding human FcγRIIa or immunogenic fragments thereof, can be expressed in cells in vitro, followed by isolation of the human FcγRIIa and administration of the FcγRIIa to a suitable host in which antibodies are raised.

Alternatively, antibodies against platelet FcγRIIa may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes tor a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Kits

The invention also provides kits for the treatment or prevention of selected patients having elevated platelet FcγRIIa that are at increased risk for a cardiovascular disease, such as myocardial infarction, stroke, coronary revascularization, cerebral vascular accident, death, or other cardiovascular endpoints, disorder or symptom thereof. In one embodiment, the kit includes an effective amount of a compound, such as methotrexate or canakinumab in unit dosage form, together with instructions for selecting a subject suffering from or susceptible to a cardiovascular disease or disorder, such as myocardial interaction, stroke, coronary revascularization, cerebral vascular accident, death, or other cardiovascular endpoints, or symptoms thereof. Such subjects are selected for treatment by determining an increased level of FcγRIIa. Optionally, such kits include Vorapaxar, Clopidogrel, Prasugrel or Ticagrelor. Optionally, such kits also include rivaroxaban or warfarin. In other embodiments, the kit comprises a sterile container which contains the compound; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments. The instructions will generally include information about the use of methotrexate or canakinumab for treatment of a selected patient having or at risk for a cardiovascular event. In other embodiments, the instructions include at least one of the following: description of the compound; dosage schedule and administration for treatment of a disease or disorder or symptoms thereof, including those of a cardiovascular nature; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit me scope of what me inventors regard as their invention.

### EXAMPLES

### Example 1: FcγRIIa and Clinical Events

The platelet expression of FcγRIIa in young healthy individuals was found to be <8,000/platelet and the platelet expression in patients with atherosclerosis was >8,000/platelet. For the patients enrolled in this study, the average (±SD) platelet expression of FcγRIIa was 11,479±2,405 (median 11,211) at baseline. It was expected that a platelet expression of FcγRIIa >11,000/platelet would distinguish patients at increased risk of subsequent cardiovascular events. The odds ratio of ischemic events at different thresholds was determined (Table 1).

TABLE 1
Odds Ratio for Cardiovascular Event (Myocardial Infarction,
Cerebral Vascular Accident, Revascularization, and Death)
Occurrence based on Platelet Expression of FCγRIIa

| Platelet FCγRIIa | Odds | 95% Confidence | |
|---|---|---|---|
| Expression | Ratio | Interval | p |
| 9,000 | 3.6 | 0.81-15.92 | 0.091 |
| 10,000 | 2.2 | 0.79-6.15 | 0.133 |
| 10,500 | 3.0 | 1.08-8.42 | 0.035 |
| 11,000 | 3.2 | 1.23-8.51 | 0.017 |

The odds ratio for each of the thresholds assessed was similar. However, this analysis confirmed that 11,000 was the threshold that best identifies patients at high and low risk of cardiovascular events based on platelet expression of FcγRIIa.

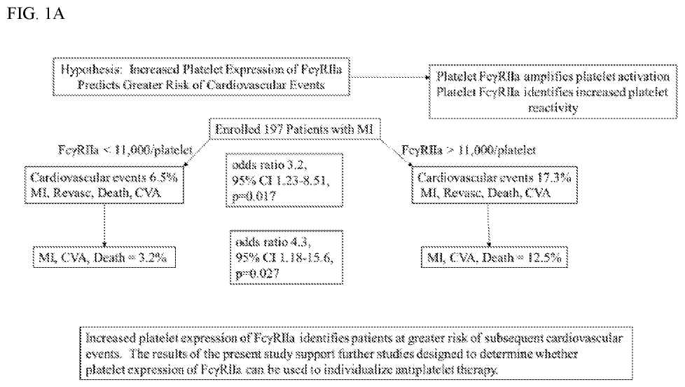

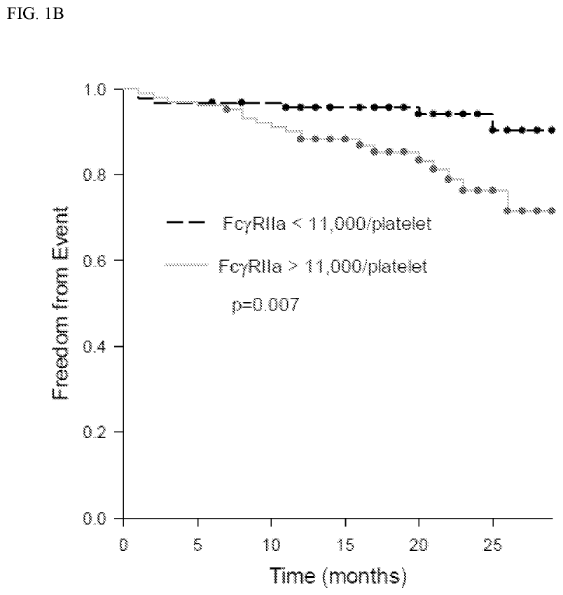

Patients with platelet expression of FcγRIIa >11,000 had a greater risk of cardiovascular events that became apparent after 6 months (FIG. 1). The difference in the incidence of cardiovascular death was driven by a greater incidence of death, MI and CVA (table 2) with revascularization being comparable in the 2 groups. The incidence of bleeding was low and comparable in the 2 groups (Table 2).

TABLE 2
Clinical Events in Patients with FCγRIIa Greater than 11,000

| | Rate | Rate | | 95% | |
|---|---|---|---|---|---|
| | (n) <11,000/plt | (n) >11,000/plt | Odds | Confidence | |
| Clinical Event | N = 93 | N = 104 | Ratio | Intervals | p |
| MI/CVA/Revasc/Death | 6.5% (6) | 17.3% (19) | 3.2 | 1.23-8.51 | 0.017 |
| MI/CVA/Death | 3.2% (3) | 12.5% (13) | 4.3 | 1.18-15.6 | 0.027 |
| MI/CVA | 3.2% (3) | 9.6% (10) | 3.2 | 0.85-12.0 | 0.085 |
| MI/Death | 3.2% (3) | 9.6% (10) | 3.2 | 0.85-12.0 | 0.085 |
| MI | 3.2% (3) | 6.7% (7) | 2.2 | 0.54-8.63 | 0.274 |
| CVA | 0 | 2.9% (3) | 6.4 | 0.33-127 | 0.220 |
| Revasc | 3.2% (3) | 3.8% (4) | 1.2 | 0.26-5.5 | 0.815 |
| Death | 0 | 2.9% (3) | 6.4 | 0.33-127 | 0.220 |
| Any Bleed | 6.5% (6) | 5.8% (6) | 0.89 | 0.28-2.85 | 0.842 |
| BARC 2 | 4.3% (4) | 5.8% (6) | 1.36 | 0.37-4.99 | 0.641 |
| BARC 3 | 2.2% (2) | 0 | 0.18 | 0.08-3.70 | 0.263 |

CVA = cerebral vascular accident,
MI = myocardial infarction,
Revasc = revascularization,
BARC = Bleeding Academic Research Consortium

### Example 2: Clinical Characteristics

Patients with platelet expression of FcγRIIa >11,000/platelet were older, more likely to have diabetes, and prior revascularization (Table 3).

TABLE 3
Patient Characteristics

| | All Patients | | FcγRIIa <11,000 | | FcγRIIa >11,000 | | |
|---|---|---|---|---|---|---|---|
| Characteristic | (n = 197) | | (n = 93) | | (n = 104) | | p |
| Age (mean ± SD) | 63 ± 12 | | 60.5 ± 11.4 | | 65.3 ± 12.2 | | 0.016 |
| Gender (% male ) | 70.6% | (139) | 71.0% | (66) | 70.2% | (73) | 0.902 |
| MI Type | | | | | | | |
| STEMI | 32.5% | (64) | 31.2% | (29) | 33.7% | (35) | 0.709 |
| NSTEMI | 67.5% | (133) | 68.8% | (64) | 66.3% | (69) | 0.709 |
| LVEF at enrollment (% ± SD) | 53.9% ± 10.7% | | 54.9% ± 8.8% | | 53.0% ± 12.1% | | 0.214 |
| HTN | 66.0% | (130) | 66.7% | (62) | 65.4% | (68) | 0.848 |
| DM | 24.4% | (48) | 17.2% | (16) | 30.8% | (32) | 0.027 |
| insulin treatment | 10.2% | (20) | 5.4% | (5) | 14.4% | (15) | 0.037 |
| Active Smoker | 25.9% | (51) | 29.0% | (27) | 23.1% | (24) | 0.347 |
| Hyperlipidemia | 67.0% | (132) | 68.8% | (64) | 65.4% | (68) | 0.613 |
| Prior MI | 22.8% | (45) | 18.3% | (17) | 26.9% | (28) | 0.152 |
| Prior Revascularization | 33.0% | (65) | 23.7% | (22) | 41.3% | (43) | 0.009 |
| CABG | 8.1% | (16) | 4.3% | (4) | 11.5% | (12) | 0.065 |
| PCI | 24.9% | (49) | 19.4% | (18) | 29.8% | (31) | 0.093 |
| PAD | 7.6% | (15) | 5.4% | (5) | 9.6% | (10) | 0.269 |
| Prior CVA | 4.6% | (9) | 4.3% | (4) | 4.8% | (5) | 0.867 |
| Renal Insufficiency | | | | | | | |
| GFR 30-59 | 6.6% | (13) | 5.4% | (5) | 7.7% | (8) | 0.518 |
| ESRD | 1.0% | (2) | 1.0% | (2) | 0% | (0) | 0.308 |
| COPD | 7.6% | (15) | 8.6% | (8) | 6.73% | (7) | 0.790 |
| Medications | | | | | | | |
| ASA | 100% | (197) | 100% | (93) | 100% | (104) | 1.000 |
| clopidogrel | 63.5% | (125) | 63.4% | (59) | 63.5% | (66) | 0.988 |
| ticagrelor | 36.5% | (72) | 36.6% | (34) | 36.5% | (38) | 0.988 |
| β-blocker | 92.4% | (182) | 92.5% | (86) | 92.3% | (96) | 0.958 |
| CCB | 13.2% | (26) | 14.0% | (13) | 12.5% | (13) | 0.757 |
| nitrates | 5.6% | (11) | 4.3% | (4) | 6.7% | (7) | 0.464 |
| ACEI/ARB | 46.2% | (91) | 46.2% | (43) | 46.2% | (48) | 1.000 |
| diuretic | 11.2% | (22) | 6.5% | (6) | 15.4% | (16) | 0.049 |
| statin | 98.5% | (194) | 96.8% | (90) | 100% | (104) | 0.067 |

MI = myocardial infarction,
STEMI = ST elevation myocardial infarction,
NSTEMI = non-ST elevation myocardial infarction,
LVEF = left ventricular ejection fraction,
HTN = hypertension,
DM = diabetes,
CABG = coronary artery bypass graft,
PCI = percutaneous coronary intervention,
PAD = peripheral arterial disease,
CVA = cerebrovascular accident,
GFR = glomerular filtration rate,
ESRD = end stage renal disease,
COPD = chronic obstructive pulmonary disease,
ASA = aspirin,
CCB = calcium channel blocker,
ACEI = angiotensin converting enzyme inhibitor,
ARB = angiotensin receptor blocker

Cox regression analysis was performed to assess the impact of these potentially confounding variables (Table 4).

TABLE 4
Cox Regression Analysis

| | Coeffi- | | | Hazard | |
|---|---|---|---|---|---|
| Covariate | cient | SE | p | Ratio | 95% CI |
| FcγRIIa >11,000/plt | 1.099 | 0.48 | 0.022 | 3.002 | 1.17-7.69 |
| Age | 0.00517 | 0.0166 | 0.756 | 1.005 | 0.97-1.04 |
| No DM | −0.188 | 0.437 | 0.667 | 0.828 | 0.35-1.95 |
| Prior Revac | 0.494 | 0.419 | 0.238 | 1.639 | 0.72-3.72 |

CI—confidence interval,
plt = platelet,
DM = diabetes mellitus,
revasc = revascularization

Platelet expression of FcγRIIa >11,000 remained significantly (hazard ratio 3.0, p=0.02) associated with a greater risk of cardiovascular events after adjustment for age, diabetes, and prior revascularization. All patients were treated with aspirin and approximately 36% were treated with ticagrelor. Treatment with clopidogrel and ticagrelor was similar in the 2 groups. Treatment with diuretic was uncommon (11% of all patients) but more prevalent in those with FcγRIIa >11,000/platelet.

### Example 3: Change in Platelet Expression of FcγRIIa

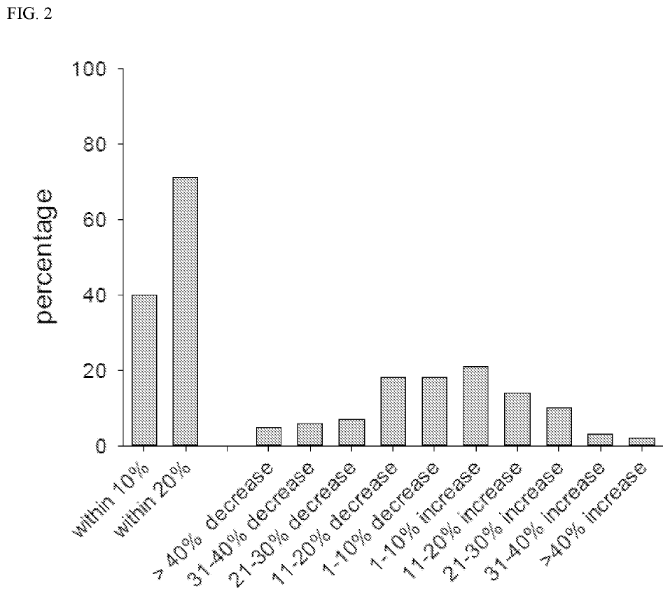

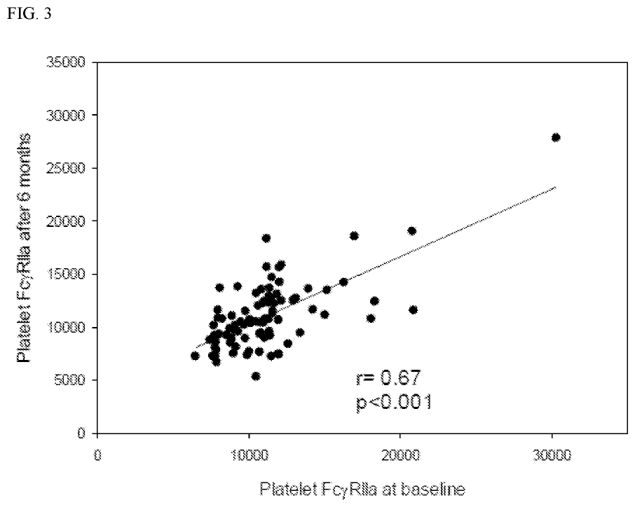

A second sample of blood was obtained to quantify platelet expression of FcγRIIa in 114 patients. Not surprisingly, platelet expression of FcγRIIa changed over time in some patients (FIG. 2). Changes were modest (<20% of the average expression) for the majority (71%) of patients. Platelet expression of FcγRIIa at baseline was a strong predictor of expression at 6 months (FIG. 3).

FcγRIIa amplifies the activation of platelets. Consistent with this observation it was found that the activation of platelets in response to multiple agonists is greater when platelet expression of FcγRIIa is high. In the present study, high platelet expression of FcγRIIa (>11,000/platelet) was associated with a greater risk (odds ratio 3.2) of subsequent cardiovascular events. A meta-analysis performed by Wisman and colleagues that assessed the clinical implications of high platelet reactivity found an odds ratio of 2.09 for obviates issues associated with the preparation of platelets that may alter platelet reactivity (Schneider et al., Circulation 1997; 96:2877-83-7, Madsen et al., Am J Cardiol 2007; 100:722-7, Lippi et al., Blood Coagul Fibrinolysis 2013; 24:666-9). Further, because platelet FcγRIIa amplifies activation (Boylan et al., Blood 2008; 112:2780-6, Lova et al. J Biol Chem 2002; 277:12009-15), greater FcγRIIa expression can be expected to reflect greater activation in response to diverse agonists and eliminates concerns regarding the appropriate agonist and concentration that should be used to assess platelet reactivity. Further, expression of FcγRIIa would not be expected to De directly influenced by currently available antiplatelet agents and so can be expected to reflect high platelet reactivity and greater risk of subsequent cardiovascular events independent of treatment. Consistent with this observation, platelet expression of FcγRIIa will not assess the adequacy of current treatment and would need to be paired with platelet function testing if demonstration of treatment effect were necessary.

Platelet expression of FcγRIIa reflects megakaryocyte production that is increased by interferon γ. Thus, platelet expression of FcγRIIa would be expected to change as a reflection of systemic inflammation. While change in platelet expression of FcγRIIa would be expected and was seen in this study, those changes would be expected to manifest over weeks to months. Currently available measures of platelet reactivity are sensitive markers of platelet function that change substantially within an individual, even during the course of a day. Thus, measurement of platelet reactivity with currently available assays is analogous to a random glucose measurement. Platelet expression of FcγRIIa may be analogous to a HbA₁C, reflecting platelet reactivity over a longer interval of time. While both measures are useful, platelet expression of FcγRIIa may be more useful to guide long term therapy.

Greater platelet expression of FcγRIIa was observed in older individuals and in patients with diabetes and previous revascularization. These observations are consistent with previous reports. The association between a greater extent of atherosclerosis and increased platelet expression of FcγRIIa is consistent with greater expression of interferon γ in association with atherosclerosis and augmentation of megakaryocyte production of FcγRIIa that is induced by interferon γ. Similarly, diabetes is associated with greater expression of FcγRIIa. Accordingly, platelet expression of FcγRIIa provides a tangible link between systemic inflammation and increased platelet reactivity.

In summary, increased platelet expression of FcγRIIa identified patients at greater risk of subsequent cardiovascular events. Because FcγRIIa augments platelet reactivity (Boylan et al., Blood 2008; 112:2780-6, Lova et al. J Biol Chem 2002; 277:12009-15), it is a novel marker of increased platelet reactivity capable of identifying increased risk of cardiovascular events. It is noteworthy that the incidence of revascularization was similar in patients with high and low platelet expression of FcγRIIa and that the difference in cardiovascular events was driven by a greater incidence of myocardial infarction, cerebrovascular accident, and death (Table 2).

### Example 4: Assessment of a Platelet Surface Protein Such as FcγRIIa has the Advantage of Simplifying Assay

Currently available measures of platelet reactivity are sensitive markers or platelet function that change substantially within an individual, even during the course of a day. Thus, measurement of platelet reactivity with currently available assays is analogous to a random glucose measurement. Platelet expression of FcγRIIa may be analogous to a HbA₁C, reflecting platelet reactivity over a longer interval of time. While both measures are useful, platelet expression of FcγRIIa may be more useful to guide long term therapy.

Greater platelet expression of FcγRIIa was observed in older individuals and in patients with diabetes and previous revascularization. The association between a greater extent of atherosclerosis and increased platelet expression of FcγRIIa is consistent with greater expression of interferon γ in association with atherosclerosis and augmentation of megakaryocyte production of FcγRIIa that is induced by interferon γ. Similarly, diabetes is associated with greater expression of FcγRIIa (Williams et al., Curr Diab Rep 2007; 7:242-248). Accordingly, platelet expression of FcγRIIa provides a tangible link between systemic inflammation and increased platelet reactivity.

In summary, it was found that increased platelet expression of FcγRIIa identifies patients at greater risk of subsequent cardiovascular events. Because FcγRIIa augments platelet reactivity (Boylan et al., Blood 2008; 112:2780-6, Lova et al. J Biol Chem 2002; 277:12009-15), it provides a novel marker of increased platelet reactivity capable of identifying increased risk of cardiovascular events. It is noteworthy that the incidence of revascularization was similar in patients with high and low platelet expression of FcγRIIa and that the difference in cardiovascular events was driven by a greater incidence of MI, CVA, and death (Table 2). Assessment of a platelet surface protein such as FcγRIIa has the advantage of simplifying assay conditions, eliminating artefactual platelet activation potentially complicating current functional assays, and being insensitive to antiplatelet treatment. Because platelet expression of FcγRIIa is likely to be a more consistent marker of increased platelet reactivity over time, it may have utility to guide individualized antiplatelet therapy. Accordingly, additional studies are indicated to determine whether platelet expression of FcγRIIa can be used to guide antiplatelet therapy.

The results described above were obtained using the following methods and materials.

Patients

All patients provided written informed consent to participate in a protocol approved by the Institutional Review Board of the University of Vermont. Inclusion criteria were a diagnosis of MI demonstrated by an elevation of troponin I greater than 0.034 ng/ml in association with clinical symptoms and demonstration of coronary artery disease on diagnostic angiography or perfusion imaging. Both ST elevation MI (STEMI) and non-ST elevation MI (NSTEMI) were included. Exclusion criteria included an active diagnosis of cancer, infection or systemic inflammatory condition as well as planned long term treatment with an anticoagulant.

Protocol

Patients were enrolled on the day of discharge of the hospitalization for MI. Clinical characteristics were recorded and a sample of blood was taken to determine platelet expression of FcγRIIa. Patients were contacted at 6±1 months, 1 year, and at the end of the study and queried for study endpoints (the query included review of records from subsequent hospitalizations). Patients were asked to have a second sample of blood taken at 6±1 months (some patients who lived remotely declined an onsite second visit). Care of the patient was not altered by participation in this trial. Phlebotomy was performed with a 21 gauge butterfly needle, tourniquets were applied for less than 90 seconds, and the first 3 ml of blood were discarded. Blood was anticoagulated with trisodium citrate (3.2%, 1:10 v/v).

Clinical Endpoints

Cardiovascular endpoints included MI, CVA, coronary revascularization, and death (not associated with bleeding). MI was diagnosed by combination of clinical symptoms and an elevation of troponin I to greater than 0.034 ng/ml. CVA was diagnosed by the combination of transient or long term symptoms plus imaging evidence of vessel occlusion and/or diagnostic findings of ischemic injury on brain imaging (intracranial bleeding was considered a bleeding event). Coronary revascularization include PCI and coronary artery bypass grafting (CABG). Patients were queried for bleeding episodes requiring medical attention. Bleeding severity was quantified in accordance with the Bleeding Academic Research Consortium (BARC) criteria (17). All clinical endpoints were reviewed independently by 2 clinicians who agreed with the designation. Clinicians were blinded to platelet expression of FcγRIIa during adjudication of clinical events.

Platelet Expression of FcγRIIa

Platelets expression of FcγRIIa was quantified with the use of flow cytometry. Whole blood was added to HEPES-Tyrode's buffer containing phycoerythin labeled anti-CD32 (Becton Dickinson Biosciences). Platelets were fixed and red cells lysed with the use of Optilyse-C (Beckman Coulter). Samples were diluted in HEPES-Tyrode's buffer to enable assessment of surface expression of FcγRIIa on individual platelets. Flow cytometric analysis was performed with the use of a Beckman Coulter Epics Elite instrument (Miami, Fla.). Platelets were identified on the basis of size (forward and side scatter). Calibration to enable quantification was accomplished with the use of Quantum simply cellular anti-mouse beads (Bangs Laboratories, Fishers, Ind.). Platelet expression of FcγRIIa was quantities wan the use of Bangs laboratories QuickCal software.

Statistical Analysis

An overall event rate of ischemic endpoints of approximately 10% was expected. The study was designed to identify a 2-fold difference in the incidence of ischemic events in patients with high compared with low platelet expression of FcγRIIa. Enrollment of 200 patients was projected to provide a power of 95% with an α<0.05. 200 patients were enrolled, however, 3 patients were lost to follow-up leaving us 197 participants.

Descriptive statistics were implemented for all measures. Comparison between high and low platelet expression of FcγRIIa was conducted with the use of 2×2 contingency table methods and Fisher's Exact test for dichotomous measures, likelihood ratio methods for 2×2 contingency tables, and two sample t-tests for continuous measures. Comparison of the incidence of clinical outcomes was examined with the use of logistic regression methods. Cox regression analysis was used to adjust for potentially confounding measures. Significance was defined as p<0.05.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating a selected subject at increased risk of a cardiovascular event, the method comprising administering to the subject an anti-inflammatory agent, wherein the subject is selected by detecting an increased level of FcγRIIa on platelets from the subject relative to a reference.

2. The method of claim 1, wherein the cardiovascular event is myocardial infarction, stroke, coronary revascularization, cerebral vascular accident, death, or another cardiovascular endpoint.

3. A method of treating a selected subject having systemic inflammation, the method comprising administering to the subject an anti-inflammatory agent, wherein the subject is selected by detecting an increased level of FcγRIIa on platelets from the subject relative to a reference.

4. The method of claim 1, wherein the agent is methotrexate or canakinumab.

5. A method of identifying a subject at increased risk of a cardiovascular event comprising:
determining a level of FcγRIIa on platelets from the subject; and
comparing the level of FcγRIIa on the platelets with a reference value, wherein an increased level compared to the reference value indicates that the subject is at increased risk of a cardiovascular event.

6. The method of claim 5, wherein the cardiovascular event is myocardial infarction, stroke, coronary revascularization, cerebral vascular accident, death, or another cardiovascular endpoint.

7. A method of identifying a subject as having systemic inflammation comprising:
determining a level of FcγRIIa on platelets from the subject; and
comparing the level of FcγRIIa on the platelets with a reference value wherein an increased level compared to the reference value indicates that the subject has systemic inflammation.

8. The method of claim 1, further comprising characterizing a biological sample of the subject for the level of IL-1beta, TNF-alpha, IL-6, C reactive protein (CRP), and/or serum amyloid A.

9. The method of claim 1, wherein the level of FcγRIIa is determined using an FcγRIIa specific reagent.

10. The method of claim 9, wherein the FcγRIIa specific reagent is an antibody or antigen-binding fragment thereof.

11. The method of claim 1, wherein the level of platelet FcγRIIa is determined using an assay selected from the group consisting of flow cytometry, immunoassay, ELISA, western blotting, and radioimmunoassay.

12. The method of claim 1, wherein the level of FcγRIIa is determined using fluorometric or colorimetric assay.

13. The method of claim 12, wherein the level of FcγRIIa is determined using flow cytometry.

14. The method of claim 1, wherein the reference value represents a level of FcγRIIa on platelets from disease-free subjects.

15. The method of claim 1, wherein the increased level is increased by at least about 1.5, 2, 3, 4, or 5-fold.

16-17. (canceled)

18. The method of claim 17, wherein the increased level of platelet FcγRIIa expression is greater than about 11,000 copies of FcγRIIa per platelet.

19-20. (canceled)

21. The method of claim 1, wherein the method further comprises administering an anti-thrombotic therapy to the selected subject.

22. The method of claim 21, wherein the anti-thrombotic therapy is one or more of prasugrel, ticagrelor, clopidogrel, and vopaxar.

23. The method of claim 1, wherein the method further comprises administering an anti-coagulant therapy to the selected subject.

24. The method of claim 23, wherein the anti-coagulant is rivaroxaban or warfarin.


SEQUENCE LISTING (3 sequences)

SEQ ID NO: 1 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 2 - DNA, 954 bp, Homo sapiens
atgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 3 - protein, 6 aa, Unknown
  Description of Unknown Protease-activated receptor (PAR) sequence
SFLLRN